(19) United States
(12) Plant Patent Application Publication
(10) Pub. No.: US 2002/0116738 P1
(43) Pub. Date: Aug. 22, 2002

(54) Spray rose plant named 'Meideskri'

(76) Inventor: Alain A. Meilland, Antibes (FR)

Correspondence Address: BURNS DOANE SWECKER & MATHIS L L P, POST OFFICE BOX 1404, ALEXANDRIA, VA, 22313-1404, US

(21) Appl. No.: 09/784,071

(22) Filed: Feb. 16, 2001

(51) Int. Cl. A01H 5/00

(52) U.S. Cl. PLT/145

(57) ABSTRACT

A new and distinct variety of rose plant of the Spray Class is provided which abundantly forms attractive double blossoms that are cream champagne in coloration. The buds are conical in configuration and commonly are borne in clusters on relatively long stems. An erect growth habit is exhibited. The foliage is dense, medium green and non-glossy and contrasts nicely with the light-colored blossoms. The plant is well suited for the production of cut floral sprays under greenhouse growing conditions.

### SUMMARY OF THE INVENTION

The new variety of Rosa hybrida spray rose plant was created by artificial pollination wherein two parents were crossed which previously had been studied in the hope that they would contribute the desired characteristics. The female parent (i.e., the seed parent) of the new variety was the ‘Kortexung’ variety (United States Plant Pat. No. 6,513). The male parent (i e., the pollen parent) of the new variety was the product of the cross of the ‘Meifota’ variety (U.S. Plant Pat. No. 6,891) and the ‘Keitaibu’ variety (U.S. Plant Pat. No. 7,739). The parentage of the new variety can be summarized as follows:

‘Kortexung’×(‘Meifota’×‘Keitaibu’).

The seeds resulting from the above pollination were sown and small plants were obtained which were physically and biologically different from each other. Selective study resulted in the identification of a single plant of the new variety.

It was found that the new variety of spray rose plant of the present invention possesses the following combination of characteristics:

(a) forms attractive conical buds on relatively long stems in the form of a spray,

(b) forms in abundance attractive double blossoms that are cream champagne in coloration,

(c) exhibits an erect growth habit, and

(d) forms attractive medium green non-glossy foliage.

The light spray inflorescence coloration contrasts nicely with the dense medium green foliage.

The new variety well meets the needs of the horticultural industry. It is particularly well-suited for use in the production of cut floral sprays under greenhouse growing conditions.

The new variety has been found to undergo asexual propagation in France by a number of routes, including budding, grafting, and cuttage. Asexual propagation by the above-mentioned methods as performed in France has shown that the characteristics of the new variety are strictly transmissible from one generation to another.

The new variety has been named the ‘Meideskri’ variety.

### BRIEF DESCRIPTION OF THE PHOTOGRAPH

The accompanying photograph shows as nearly true as it is reasonably possible to make the same in a color illustration of this character, typical specimens of the plant parts of the new variety. The rose plants of the new variety were two years of age were observed during January while growing on Rosa indica understock in greenhouses at LeCannet des Maures, Var, France. Dimensions in centimeters are indicated at the bottom of the photograph.

FIG. 1—illustrates a specimen of a young shoot;

FIG. 2—illustrates a specimen of a floral bud before the opening of the sepals;

FIG. 3—illustrates a specimen of a floral bud at the opening of the sepals;

FIG. 4—illustrates a specimen of a floral bud at the opening of the petals;

FIG. 5—illustrates a specimen of a flower during the course of opening;

FIG. 6—illustrates a specimen of an open flower—plan view obverse;

FIG. 7—illustrates a specimen of an open flower—plan view reverse;

FIG. 8—illustrates a specimen of a fully open flower—plan view—obverse;

FIG. 9—illustrates a specimen of a fully open flower—plan view—reverse;

FIG. 10—illustrates a specimen of a floral receptacle showing the arrangement of the stamens and pistils;

FIG. 11—illustrates a specimen of a floral receptacle showing the arrangement of the pistils (stamens removed);

FIG. 12—illustrates a specimen of a flowering stem;

FIG. 13—illustrates a specimen of a main branch;

FIG. 14—illustrates a specimen of a leaf with three leaflets—plan view—upper surface;

FIG. 15—illustrates a specimen of a leaf with five leaflets—plan view—under surface;

FIG. 16—illustrates a specimen of a stem with one flower; and

FIG. 17—illustrates a specimen of a stem with a cluster of buds and a flower.

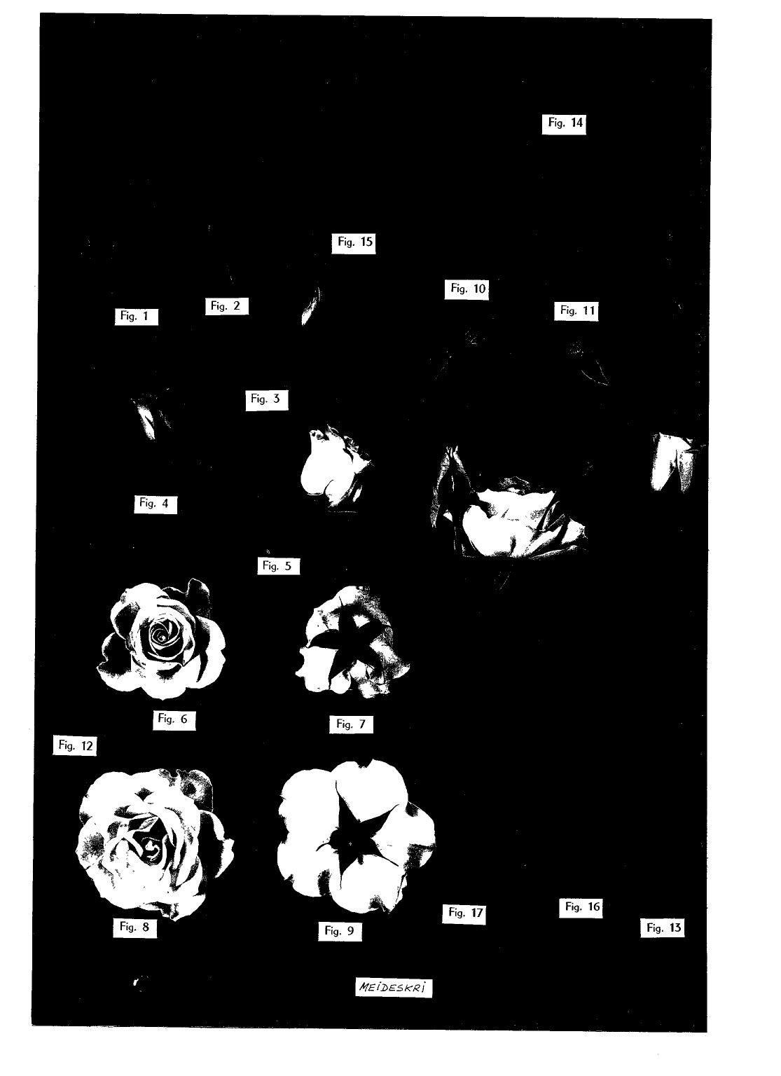

### DETAILED DESCRIPTION

The chart used in the identification of the colors is that of The Royal Horticultural Society (R.H.S. Colour Chart). When more common color terms are utilized they are to be accorded customary dictionary significance. The description is based on two year-old specimens of the new variety which were observed during November while budded on Rosa indica understock and growing in greenhouses at LeCannet des Maures, Var, France.

Class:

Spray.—

Plant:

Height.—a plant pruned to a height of 85 cm commonly produces floral stems having a length of approximately 40 to 60 cm.

Habit.—erect.

Branches:

Color.—young stems: dark green, near Green Group 138A. adult wood: dark green, near Green Group 138A.

Thorns.—size: generally small (as illustrated). quantity: few on flowering stems and numerous on adult wood (as illustrated). configuration: very fringed on the upper surface and slightly concave on the under surface. color: pinkish on young stems and pinkish to tan on adult wood, near Greyed-Orange Group 171A (as illustrated).

Leaves:

Petioles.—rather glandular with rare prickles.

Stipules.—adnate, pectinate, and rather broad.

Leaflets.—number: 3, 5 (most often), and 7. shape: generally oval with a narrow generally symmetrical tip and a generally rounded base (as illustrated). texture: typical and normal. serration: regular. general appearance: dense, medium green, and dull foliage. color (young foliage): upper surface: near Green Group 137A (as illustrated). under surface: near Yellow-Green Group 148A. color (adult foliage): upper surface: near Green Group 136A. under surface: near Yellow-Green Group 148A.

Inflorescence

Number of flowers.—commonly approximately 2 to 5 per stem.

Peduncle.—near Yellow-Green Group 146A in coloration, (as illustrated), and the length is approximately 7 cm on average.

Sepals.—light green and tomentose on the upper surface, light green and glabrous on the under surface, and commonly without extensions (as illustrated).

Buds.—shape: conical. length: approximately 2.5 cm on average. size: small.

Color.—upper surface: Orange Group 24D, suffused and bordered with Orange Group 25D. under surface: near Yellow-Orange Group 20C, suffused and bordered with Yellow-Orange Group 23D.

Flower.—form: double and cup-shaped. diameter: approximately 8 cm on average. color (when opening begins): upper surface: Yellow-Orange Group 19D on the external petals, and Yellow-Orange Group 20C and very lightly suffused with Orange Group 27B on the internal petals. under surface: Yellow-Orange Group 16D. color (when blooming): upper surface: Yellow-Orange Group 19D on the external petals, and Yellow-Orange Group 20C and very lightly suffused with Orange Group 27B on the internal petals. under surface: Yellow-Orange Group 16D. color (at end of opening): upper surface: Yellow-Orange Group 19D and lightly suffused with Orange Group 27B and 27C on the internal petals, and Yellow-Orange Group 19D on the external petals. under surface: Yellow-Orange Group 19D. fragrance: none. petal configuration: rounded base and a reflexed tip. petal drop: good, the petals commonly detach cleanly. anthers: yellow in coloration. stamen number: approximately 150 on average. filaments: yellow in coloration. pistil number: approximately 100 on average. stigmas: yellow in coloration. styles: greenish in coloration. receptacle: light green in coloration, and in longitudinal section in the shape of a funnel. lasting quality: very long, approximately 8 to 10 days on the plant, and approximately 8 to 12 days when cut and placed in a vase.

Development

Vegetation.—strong.

Blooming.—abundant.

I claim:

1. A new and distinct variety of Rosa hybrida spray rose plant characterized by the following combination of characteristics:
(a) forms attractive conical buds on relatively long stems in the form of a spray,
(b) forms in abundance attractive double blossoms that are cream champagne in coloration,
(c) exhibits an erect growth habit, and
(d) forms attractive medium green non-glossy foliage; substantially as herein shown and described.
